(19) United States
(12) Patent Application Publication
(10) Pub. No.: US 2022/0084677 A1
(43) Pub. Date: Mar. 17, 2022

(54) SYSTEM AND METHOD FOR GENERATING DIFFERENTIAL DIAGNOSIS IN A HEALTHCARE ENVIRONMENT

(71) Applicant: Novocura Tech Health Services Private Limited, Bangalore (IN)

(72) Inventors: Krishnam GUPTA, Uttar Pradesh (IN); Jaiprasad RAMPURE, Bidar (IN); Hunaif MUHAMMAD, Calicut (IN); Vamsi ADARI, Visakhapatnam (IN); Monu KRISHNAN, Thiruvananthapuram (IN); Subbarao Nikhil NARAYAN, Bengaluru (IN); Ajit Kumar NARAYANAN, Bengaluru (IN)

(21) Appl. No.: 17/108,348

(22) Filed: Dec. 1, 2020

(30) Foreign Application Priority Data
Sep. 14, 2020 (IN) 202041039775

(51) Int. Cl. G16H 50/20; G06N 3/08; G06N 3/04

(52) U.S. Cl. CPC G16H 50/20; G06N 3/04; G06N 3/08

(57) ABSTRACT

A system for generating a differential diagnosis in a healthcare environment is presented. The system includes a receiver configured to receive one or more user inputs and generate a plurality of input streams. The system further includes a processor including a multi-stream neural network, a training module, and a differential diagnosis generator. The multi-stream neural network includes a plurality of feature extractor sub-networks and a combiner sub-network. The training module includes a feature optimizer configured to train each feature-extractor sub-network individually, and a combiner optimizer configured to train the plurality of feature extractor sub-networks and the combiner sub-network together. The training module is configured to alternate between the feature optimizer and the combiner optimizer until a training loss reaches a defined saturation value. The differential diagnosis generator is configured to generate the differential diagnosis based on a combined feature set generated by the trained multi-stream network.

### PRIORITY STATEMENT

The present application claims priority under 35 U.S.C. § 119 to Indian patent application number 202041039775 filed Sep. 14, 2020, the entire contents of which are hereby incorporated herein by reference.

### BACKGROUND

Embodiments of the present invention generally relate to systems and methods for generating a differential diagnosis in a healthcare environment using a trained multi-stream neural network, and more particularly to systems and methods for generating a differential diagnosis of a dermatological condition using a trained multi-stream neural network.

Typical commercial approaches for automated differential diagnosis in a healthcare domain consider inputs from a single modality/signal. For example, such approaches typically use either text or images alone. The accuracy of such approaches to generate a differential diagnosis is therefore low. Further, conventional approaches that include inputs from multiple modalities/signals may not consider the correlation between the different signals.

Moreover, for certain automated healthcare diagnosis (such as dermatological diagnosis), a user (such as the patient or the caregiver) typically provides a user-grade image that are usually captured using imaging devices such as a mobile camera or a hand held digital camera. A user-grade image, however, may have high background variance, high lighting variation, no fixed orientation and/or no fixed scale. Thus, inferring a diagnosis from such images may be usually difficult.

Thus, there is a need for systems and methods capable of generating differential diagnosis by processing different types of signals together. Further, there is a need for systems and methods capable of generating differential diagnosis by learning the correlation between different type of input signals. Furthermore, there is a need for systems and methods capable of generating differential diagnosis from a user-grade image.

### SUMMARY

The following summary is illustrative only and is not intended to be in any way limiting. In addition to the illustrative aspects, example embodiments, and features described, further aspects, example embodiments, and features will become apparent by reference to the drawings and the following detailed description.

Briefly, according to an example embodiment, a system for generating a differential diagnosis in a healthcare environment is presented. The system includes a receiver and a processor operatively coupled to the receiver. The receiver is configured to receive one or more user inputs and generate a plurality of input streams based on the one or more user inputs. The processor includes a multi-stream neural network, a training module, and a differential diagnosis generator. The multi-stream neural network includes a plurality of feature extractor sub-networks configured to generate a plurality of feature sets, each feature extractor sub-network configured to generate a feature set corresponding to a particular input stream. The multi-stream neural network includes a combiner sub-network configured to generate a combined feature set based on the plurality of features sets. The training module is configured to generate a trained multi-stream neural network. The training module includes a feature optimizer configured to train each feature-extractor sub-network individually, and a combiner optimizer configured to train the plurality of feature extractor sub-networks and the combiner sub-network together. The training module is configured to alternate between the feature optimizer and the combiner optimizer until a training loss reaches a defined saturation value. The differential diagnosis generator is configured to generate the differential diagnosis based on a combined feature set generated by the trained multi-stream network.

According to another example embodiment, a system for generating differential diagnosis for a dermatological condition is presented. The system includes a receiver and a processor operatively coupled to the receiver. The receiver is configured to receive a user-grade image of the dermatological condition, the receiver further configured to generate a global image stream and a local image stream from the user-grade image. The processor includes a dual-stream neural network, a training module, and a differential diagnosis generator. The dual-stream network neural includes a first feature extractor sub-network configured to generate a plurality of global feature sets based on the global image stream. The dual-stream neural network further includes a second feature extractor sub-network configured to generate a plurality of local feature sets based on the local image stream. The dual-stream neural network furthermore includes a combiner sub-network configured to generate the combined feature set based on the plurality of global feature sets and the plurality of local feature sets. The training module is configured to generate a trained dual-stream neural network. The training module includes a feature optimizer configured to train the first feature extractor sub-network and the second feature extractor sub-network individually. The training module further includes a combiner optimizer configured to train the first feature extractor sub-network the second feature extractor sub-network, and the combiner optimizer together. The training module is configured to alternate between the feature optimizer and the combiner optimizer until a training loss reaches a defined saturation value. The differential diagnosis generator is configured to generate the differential diagnosis of the dermatological condition, based on a combined feature set generated by the trained dual-stream network.

According to another example embodiment, a method for generating a differential diagnosis in a healthcare environment is presented. The method includes training a multi-stream neural network to generate a trained multi-stream neural network, the multi-stream network includes a plurality of feature extractor sub-networks and a combiner sub-network. The training includes training each feature-extractor sub-network individually, training the plurality of feature extractor sub-networks and the combiner sub-network together, and alternating between training each feature-extractor sub-network individually and training the plurality of feature extractor sub-networks and the combiner sub-network together, until a training loss reaches a defined saturation value, thereby generating a trained multi-stream neural network. The method further includes generating a plurality of input streams from one or more user inputs and presenting the plurality of input streams to the trained multi-stream neural network. The method furthermore includes generating a plurality of feature sets from the plurality of feature extractor sub-networks of the trained multi-stream neural network, based on the plurality of input streams. Moreover, the method includes generating a combined feature set from the combiner sub-network of the trained multi-stream neural network, based on the plurality of features sets. The method further includes generating the differential diagnosis based on the combined feature set.

### BRIEF DESCRIPTION OF THE FIGURES

These and other features, aspects, and advantages of the example embodiments will become better understood when the following detailed description is read with reference to the accompanying drawings in which like characters represent like parts throughout the drawings, wherein:

FIG. 1 is a block diagram illustrating an example system for generating a differential diagnosis in a healthcare environment, according to some aspects of the present description,

FIG. 2 is a block diagram illustrating an example multi-stream neural network, according to some aspects of the present description,

FIG. 3 is a block diagram illustrating an example dual-stream neural network, according to some aspects of the present description,

FIG. 4 is a block diagram illustrating an example system for generating a differential diagnosis of a dermatological condition, according to some aspects of the present description,

FIG. 5 is a block diagram illustrating an example dual-stream neural network for generating a differential diagnosis of a dermatological condition, according to some aspects of the present description,

FIG. 6 is flow chart for a method of generating a differential diagnosis in a healthcare environment, according to some aspects of the present description,

FIG. 7 is a flow chart for a method step of FIG. 6, according to some aspects of the present description,

FIG. 8 illustrates a process flow for generating a local image from a user-grade image of a skin-disease using weakly supervised segmentation maps, according to some aspects of the present description,

FIG. 9 is a block diagram illustrating the architecture of an example dual-stream neural network, according to some aspects of the present description,

FIG. 10 is a graph showing the performance of a dual-stream neural network for two different data sets over multiple phases of optimization, according to some aspects of the present description, and

FIG. 11 is a block diagram illustrating an example computer system, according to some aspects of the present description.

### DETAILED DESCRIPTION OF EXAMPLE EMBODIMENTS

Various example embodiments will now be described more fully with reference to the accompanying drawings in which only some example embodiments are shown. Specific structural and functional details disclosed herein are merely representative for purposes of describing example embodiments. Example embodiments, however, may be embodied in many alternate forms and should not be construed as limited to only the example embodiments set forth herein. On the contrary, example embodiments are to cover all modifications, equivalents, and alternatives thereof.

The drawings are to be regarded as being schematic representations and elements illustrated in the drawings are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

Before discussing example embodiments in more detail, it is noted that some example embodiments are described as processes or methods depicted as flowcharts. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figures. It should also be noted that in some alternative implementations, the functions/acts/steps noted may occur out of the order noted in the figures. For example, two figures shown in succession may, in fact, be executed substantially concurrently or may sometimes be executed in the reverse order, depending upon the functionality/acts involved.

Further, although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or a section. Thus, a first element, component, region, layer, or section discussed below could be termed a second element, component, region, layer, or section without departing from the scope of example embodiments.

Spatial and functional relationships between elements (for example, between modules) are described using various terms, including “connected,” “engaged,” “interfaced,” and “coupled.” Unless explicitly described as being “direct,” when a relationship between first and second elements is described in the description below, that relationship encompasses a direct relationship where no other intervening elements are present between the first and second elements, and also an indirect relationship where one or more intervening elements are present (either spatially or functionally) between the first and second elements. In contrast, when an element is referred to as being “directly” connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular example embodiments only and is not intended to be limiting. Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

Unless specifically stated otherwise, or as is apparent from the description, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

Example embodiments of the present description provide systems and methods for generating a differential diagnosis in a healthcare environment using a trained multi-stream neural network. Some embodiments of the present description provide systems and methods for generating a differential diagnosis of a dermatological condition using a trained multi-stream neural network.

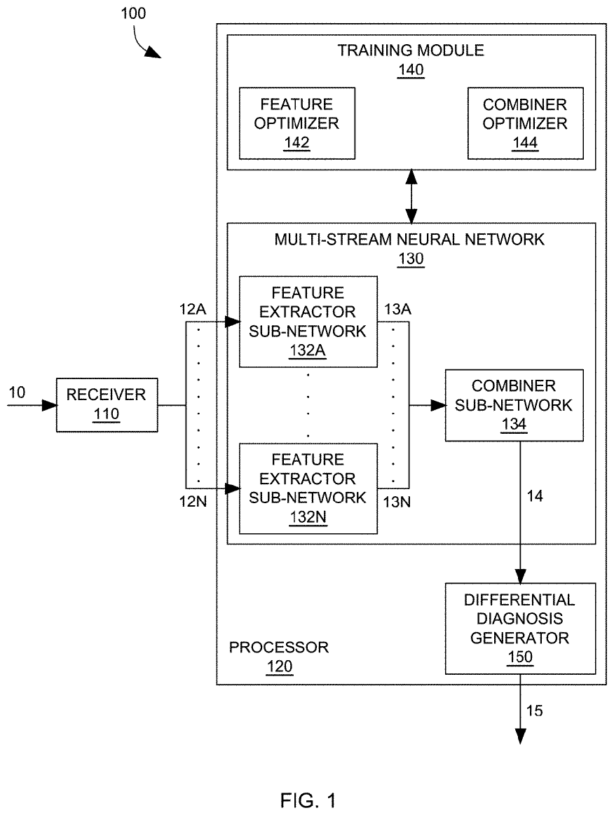

FIG. 1 illustrates an example system 100 for generating a differential diagnosis in a healthcare environment, in accordance with some embodiments of the present description. The system 100 includes a receiver 110 and a processor 120.

The receiver 110 is configured to receive one or more user inputs 10 and generate a plurality of input streams (12A . . . 12N) based on the one or more user inputs 10. User may be a patient with a health condition requiring diagnosis, or a caregiver for the patient. The one or more user inputs 10 may include a text input, an audio input, an image input, a video input, or combinations thereof.

Non-limiting examples of text inputs may include one or more symptoms observed by the user, lab report results, or some other meta information such as duration of the symptoms, location of affected body parts, and the like. Non-limiting examples of image inputs may include user-grade images, clinical images, X-ray images, ultrasound images, MM scans and the like. The term “user-grade” image as used herein refers to images that have been captured by the users themselves. These images are usually captured using imaging devices such as a mobile camera or a hand held digital camera. Non-limiting examples of audio inputs may include heart sounds, lung sounds, breathing sounds, cough sounds, and the like. Similarly, video inputs may include movement of a particular body part, video of an affected body part from different angles and/or directions, and the like.

In some embodiments, the one or more user inputs 10 include a plurality of inputs, each input of the plurality of inputs corresponding to a different modality. The term “modality” as used herein refers to the format of the user input. For example, a text input is of a different modality from a video input. Similarly, an audio input is of a different modality from an image input. In some embodiments, the one or more user inputs 10 include a plurality of inputs, wherein at least two inputs of the plurality of inputs correspond to the same modality. The user input in some instances may also include a single input from two different modalities. For example, an X-ray image that also includes text.

In some embodiments, the one or more user inputs 10 include a single input and the plurality of input streams include different input streams corresponding to the single input 10. By way of example, the single input may include a user-grade image of an affected body part with the condition to be diagnosed. As mentioned earlier, these images may have high background variance, high lighting variation, no fixed orientation and/or no fixed scale. Thus, inferring a diagnosis from such images may be usually difficult. Embodiments of the present description address the challenges associated with inferring a diagnosis using these user-grade images by first generating a plurality of input streams from the user-grade image (as described in detail later) and employing a multi-stream neural network to process these images.

The receiver 110 may be configured to generate the plurality of input streams corresponding to the modalities of the user inputs, or alternately, may be configured to generate the plurality of input streams based on the same input modality. For example, for user inputs 10 include an image input, an audio input, and a text input, the receiver 110 may be configured to generate an image input stream 12A, an audio input stream 12B, and a text input stream 12C. Alternately, the receiver 110 may be configured to generate a first image input stream 12A and a second image input stream 12B based on the image input, a first audio input stream 12C and a second audio input stream 12D based on the audio input, and a text input stream 12E based on the text input. The number and type of inputs streams generated by the receiver may depend on the user inputs and the multi-stream neural network architecture. The number of inputs streams may be in a range from 2 to 10, in a range from 2 to 8, or in a range from 2 to 6, in some embodiments.

Referring again to FIG. 1, the system 100 further includes a processor 120 operatively coupled to the receiver 110. The processor 120 includes a multi-stream neural network 130, a training module 140 and a differential diagnosis generator 150. Each of these components are described in detail below.

The term “multi-stream neural network” as used herein refers to a neural network configured to process two or more than two input steams. The multi-stream neural network 130 includes a plurality of feature extractor sub-networks 132A . . . 132N configured to generate a plurality of feature sets 13A . . . 13N, as shown in FIG. 1. Each feature extractor sub-network is configured to generate a feature set corresponding to a particular input stream. The architecture of the feature extractor sub-network is determined based on the input stream type from which the features need to be extracted. Thus, by way of example, architecture of a feature extractor sub-network for a text-based input stream would be different from a feature extractor sub-network for a video-based input stream. Further, the number of feature extractor sub-networks in the multi-stream neural network 130 is determined by the number of input streams 12A . . . 12N. The multi-stream neural network 130 further includes a combiner sub-network 134 configured to generate a combined feature set 14 based on the plurality of features sets 13A . . . 13N.

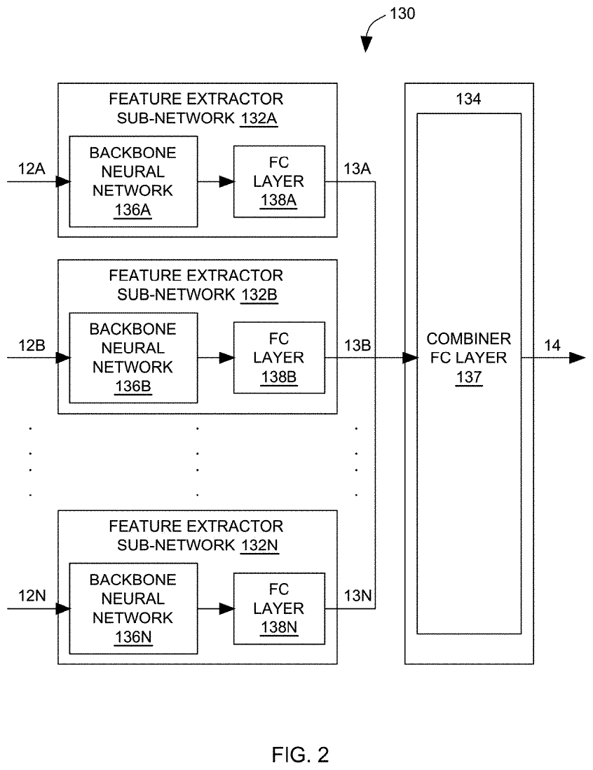

FIG. 2 is a block diagram illustrating the architecture of the multi-stream neural network 130 in more details. As shown in FIG. 2, each feature extractor sub-network 132A, 132B . . . 132N includes a backbone neural network 136A, 136B . . . 136N and a fully connected (FC) layer 138A, 138B . . . 138N. The FC layers of all the feature-extractor sub-networks are concatenated together to form a combiner FC layer 137 of the combiner sub-network 134.

The backbone neural network 136A, 136B . . . 136N works as a feature extractor and is selected based on the input steam that it is supposed to be processed. Non-limiting examples of a backbone neural network configured to generate the plurality of feature sets from an image-based input stream includes Resnet50, Resnet101, InceptionV3, MobileNet, or ResNeXt. Non-limiting examples of a backbone neural network configured to generate the plurality of feature sets from a text-based input stream include BERT, GPT, ulmfit, or T5. Non-limiting examples of a backbone neural network configured to generate the plurality of feature sets from an audio-based input steam include MFCC or STFT. Further, non-limiting examples of a backbone neural network configured to generate the plurality of feature sets from a video-based input stream include I3D or LRCN.

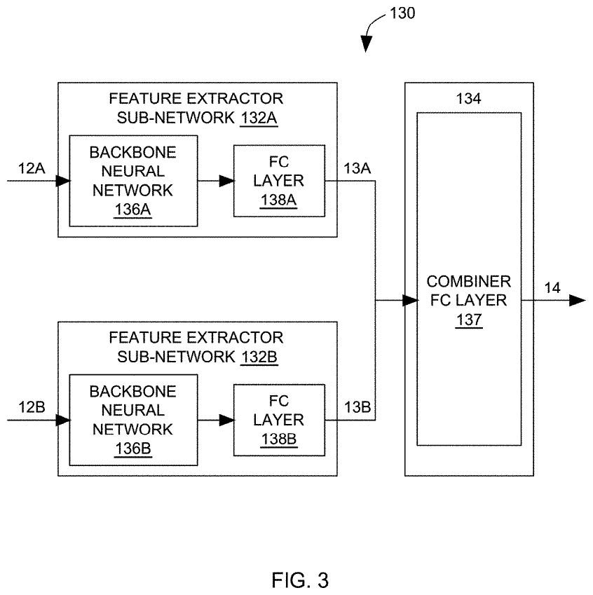

In some embodiments, the multi-stream network 130 of FIG. 1 is a dual-stream neural network 130, as shown in FIG. 3. In the embodiment illustrated in FIG. 3, the input streams 12A and 12B may correspond to text-based input stream and image-based input stream, respectively. In such instances, the backbone neural network 136A may be selected for a text stream and the backbone-neural network 136B may be selected for an image stream 12. Similarly, the input streams 12A and 12B may correspond to text-based input stream and audio-based input stream, respectively. In such instances, the backbone neural network 136A may be selected for a text stream and the backbone-neural network 136B may be selected for an audio-based stream. In some other embodiments, the input streams 12A and 12B may correspond to image-based input stream and audio-based input stream, respectively. In such instances, the backbone neural network 136A may be selected for an image stream and the backbone-neural network 136B may be selected for an audio stream. In certain embodiments, as described in detail later, both the input stream 12A and 12B may correspond to image-based input streams, and the backbone neural networks 136A and 136B may be selected for image streams.

Referring back to FIG. 1, the processor 120 further includes a training module 140 configured to train the multi-stream neural network 130 and to generate a trained multi-stream neural network. The training module 140 is configured to train the multi-stream neural network 130 using a training data set that may be presented to the receiver 110. The receiver 110 may generate a plurality of training input streams (not shown in Figures) based on the one or more inputs received from the training data set. The plurality of feature extractor sub-networks 132A . . . 132N and the combiner sub-network 134 are configured to receive and process the plurality of training input streams in a manner similar to the plurality of input streams 12A . . . 12N, as described earlier.

As shown in FIG. 1, the training module 140 includes a feature optimizer 142 configured to train each feature-extractor sub-network of the plurality of feature extractor sub-networks 132A . . . 132N individually. The training module 140 further includes a combiner optimizer 144 configured to train the plurality of feature extractor sub-networks 132A . . . 132N and the combiner sub-network 134 together.

Each feature extractor sub-network has a loss associated with it (L_{FE}). The loss (L_{FE}) for each feature extractor sub-network is based on the type of input stream that feature extractor sub-network is configured to process. The feature optimizer 142 is configured to train each feature-extractor sub-network individually by optimizing each feature-extractor sub-network loss (L_{FE}).

Further, the combiner sub-network also has a loss (L_{C}) associated with it based on the concatenated input stream. The combiner optimizer 144 is configured to train the plurality of feature extractor sub-networks 132A . . . 132N and the combiner sub-network 138 together by optimizing a total loss (L_{T}). A total loss (L_{T}) may be obtained by combining the loss from each feature-extractor sub-network (L_{FE}) and a combiner loss (L_{C}). In some embodiments, total loss (L_{T}) may be obtained by adding the combiner loss (L_{C}) to the losses from each feature-extractor sub-network (L_{FE}) multiplied by a hyper-parameter called loss ratio (β)

In accordance with embodiments of the present description, the training module 140 is configured to alternate between the feature optimizer 142 and the combiner optimizer 144 until a training loss reaches a defined saturation value. Thus, the training of the multi-stream neural network 130 is implemented in a round robin manner by first training the individual feature extractor sub-networks 132A . . . 132N individually, followed by the training the full network including the combiner sub-network 134. The individual feature extractor sub-networks may be either trained simultaneously o sequentially.

The training of the multi-stream neural network 130 in a round-robin manner is continued until the learning plateaus. In accordance with some embodiments of the present description, plateauing of the learning may be determined based on a defined saturation value of the training loss. Thus, when an overall training loss reached a defined saturation value, the training may be stopped and a trained neural network may be generated.

The training module 140 is configured to alternate between the feature optimizer 142 and the combiner optimizer 144 “n” times. Thus, the term “n” as used herein also refers to the number of rounds of training that the multi-stream neural network 130 is subjected to. In some embodiments, “n” is in a range from 1 to 5. In some embodiments, “n” is in a range from 2 to 4. It should be noted that when “n” is 1, the training module 140 is configured to stop the training of the multi-stream neural network 130 after the first round of training.

Similarly, when “n” is 2, the training module is configured to alternate between the feature optimizer 142 and the combiner optimizer 144 twice. That is, in the first round, the feature optimizer 142 optimizes each feature-extractor sub-network individual followed by the combiner optimizer 144 optimizing the plurality of feature extractor sub-networks and the combiner sub-network together. After the completion of the first round, the feature optimizer 142 again optimizes each feature-extractor sub-network individual followed by the combiner optimizer 144 optimizing the plurality of feature extractor sub-networks and the combiner sub-network together.

Once the training of the multi-stream neural network is completed, a trained multi-stream neural network is generated. The trained multi-stream neural network is configured to receive the plurality of input streams 12A . . . 12N from the receiver 110 and generate a combined feature set 14.

As shown in FIG. 1, the processor 120 further includes a differential diagnosis generator 150. The differential diagnosis generator 150 is configured to generate the differential diagnosis based on a combined feature set 14 generated by the trained multi-stream network.

According to embodiments of the present invention, each individual feature extractor sub-network 132A . . . 132N learns relevant features from the respective input stream. The combiner FC layer 137 and associated loss helps in learning complementary information from the multiple streams and improve accuracy. The round robin optimization strategy makes the multi-stream neural network 130 more efficient by balancing learning between the independent features and the complementary features of the input streams. Thus, embodiments of the present invention provide improved differential diagnosis as knowledge from the different streams (and modalities if the one or more or more inputs are of different modalities) can be combined efficiently to improve the training of the multi-stream neural network 130, which in turn results in better diagnosis.

As noted earlier, a differential diagnosis system according to embodiments of the present description is not only configured to process input stream of different modalities, but also when the input streams are from the same modality, as long as they have some complementary information to learn from.

In some embodiments, the system according to embodiments of the present description is configured to generate a differential diagnosis from a user-grade image. In certain embodiments, the system according to embodiments of the present description is configured to generate a differential diagnosis of a dermatological condition from a user-grade image using a dual-stream neural network.

As mentioned earlier, user-grade images may have high background variance, high lighting variation, no fixed orientation and/or no fixed scale. Thus, inferring a diagnosis from such images may be usually difficult. Further, as the size of a neural network is directly proportional to the size of the image, an image presented to the neural network is typically down sampled to a low resolution. This poses a challenge in learning localized features (e.g., locating small sized modules in chest X-Ray images, or locating small sized dermatological condition from a user-grade image) where the ratio of abnormality size to image size can be very small. Typical approaches to address this problem employ patch-based algorithms. However, these approaches lack the global context which is available in the full image and thus may miss out on some key information like relative size of the anomaly, or relative location of the anomaly when viewed at a higher scale.

Embodiments of the present description address the challenges associated with inferring a diagnosis using these user-grade images by first generating a global image input stream and a local image input stream from the user-grade image, and employing a dual-stream neural network to process these images.

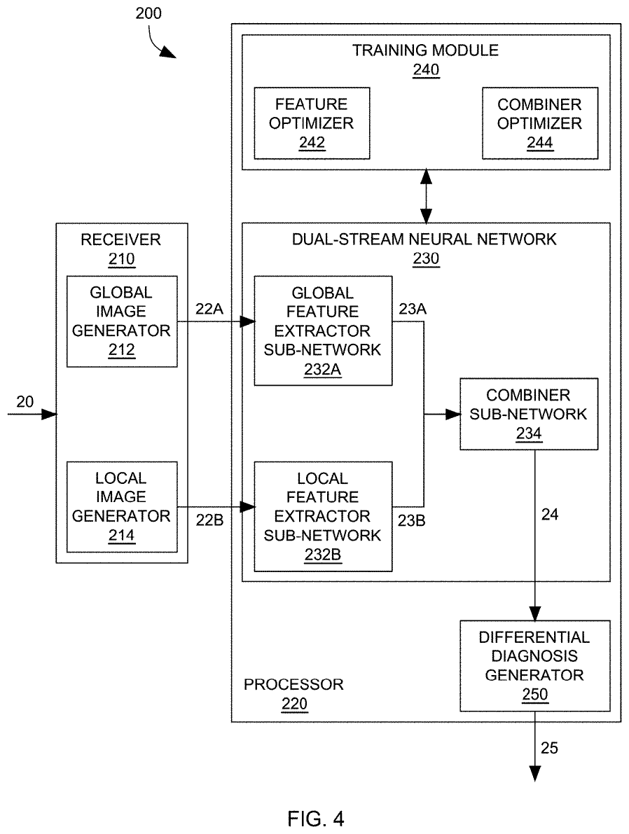

FIG. 4 illustrates an example system 200 for generating a differential diagnosis of a dermatological condition using a dual-stream neural network 230, in accordance with some embodiments of the present description. The system 200 includes a receiver 210 and a processor 220.

The receiver 210 is configured to receive a user-grade image 10 of the dermatological condition. The receiver is further configured to generate a global image stream 12A and a local image stream 12B from the user-grade image 10. As shown In FIG. 4, the receiver 210 may further include a global image generator 212 configured to generate the global image stream 12A from the user-grade image 10 by down sampling the user-grade image 10. The receiver 210 may further includes a local image generator 214 configured to generate the local image stream 12B from the user-grade image 10 by extracting regions of interest from the user-grade image 10.

The term “global image” as used herein refers to the full image of the dermatological condition as provided by the user. However, as noted earlier, the desired resolution of an input image to the neural network is low as the size of the neural network is directly proportional to the image resolution. Therefore, the term “global image stream” as used herein refers to a down sampled image stream generated from the user-grade image.

The term “local image” as used herein refers to a localized patch of the regions of interest extracted from the user-grade image. In some embodiments, the local image may also be down sampled to the same resolution as the global image to generate the local image stream. Therefore, the term “local image stream” as used herein refers to a down sampled image stream generated from a localized patch image generated from the user-grade image. The method of extracting the localized patch image based on the regions of interest is described in detail later.

The processor 220 includes a dual-stream neural network 230, a training module 240 and a differential diagnosis generator 250. The general operation of these components is similar to components described herein earlier with reference to FIG. 1.

As shown in FIG. 4, the dual-stream neural network 230 includes a first feature extractor sub-network 232A configured to generate a plurality of global feature 23A sets based on the global image stream 22A. The dual-stream neural network 230 further includes a second feature extractor sub-network 232B configured to generate a plurality of local feature sets 23B based on the local image stream 22B. The dual-stream neural network 230 further includes a combiner sub-network 234 configured to generate a combined feature set 24 based on the plurality of global feature sets 23A and the plurality of local feature sets. 23B.

The term “global features” as used herein refers to high-level features like shape, location, object boundaries of the dermatological condition, and the like, whereas the term “local features” as used herein refers to low-level information like textures, patch features, and the like. Global features, such as the location of the lesion, are important for classification of those diseases that always occur in the same location. For instance, Tinea Cruris always occurs on the inner side of thigh and groin, Palmar Hyperhydrosis always occurs on the palm of hand, Tinea Pedis and Plantar warts always occur in the feet. Local features on the other hand play an important role in the diagnosis of conditions like Nevus, Moles, Furuncle, Urticaria etc., where the lesion area might be quite small, and it may not be properly visible in the full-sized user-grade image.

To take advantage of both global and local features, a two-level image pyramid is employed in the dual-stream neural network 230 according to embodiments of the present description. A down-sampled user-grade image 22A is used to extract global features while a localized patch image 22B of the region of interest (generated using segmentation masks) is used to extract local features. The combiner sub-network 234 learns the correlation between the global features and the local features, and thus the dual-stream neural network 230 uses both the global and the local feature to classify the skin condition of interest.

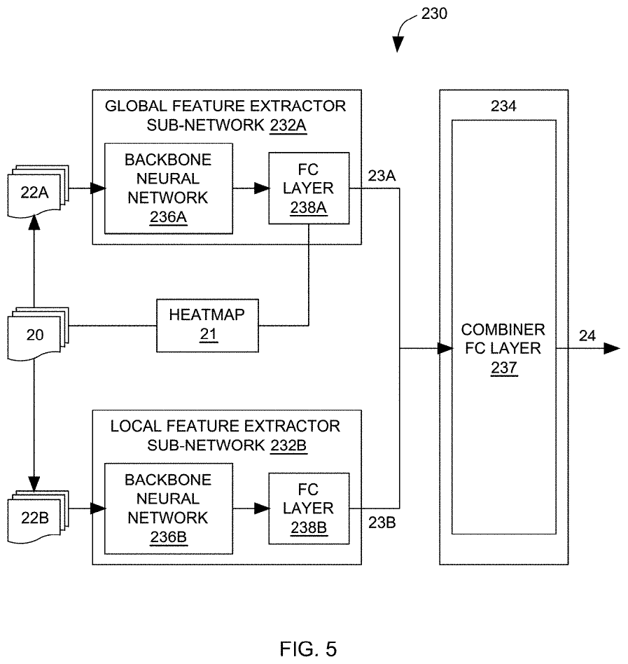

FIG. 5 is a block diagram illustrating the architecture of the dual-stream neural network 230 in more details. As shown in FIG. 5, the feature extractor sub-networks 232A and 232B includes a backbone neural network 236A and 256B, respectively; and a fully connected (FC) layer 238A and 238N, respectively. The FC layers of both the feature-extractor sub-networks are concatenated together to form a combiner FC layer 237 of the combiner sub-network 234. Non-limiting example of backbone neural networks 236A and 236B configured to generate the plurality of feature sets from an image-based input stream includes a Resnet50 neural network. Further, as shown In FIG. 5, the local image stream 23B is generated by extracting regions of interest using a heat map from the user-grade image 20.

The processor 220 further includes a training module 240 including a feature optimizer 242 and a combiner optimizer 244. The feature optimizer 242 is configured to train the first feature extractor sub-network 232A and the second feature extractor sub-network individually 232B. The combiner optimizer 244 is configured to train the first feature extractor sub-network 232A, the second feature extractor sub-network 232B, and the combiner optimizer together 234.

Each feature extractor sub-network has a loss associated with it (L_{FE}). The feature optimizer 242 is configured to train each feature-extractor sub-network individually by optimizing each feature-extractor sub-network loss (L_{FE}). Further, the combiner sub-network also has a loss (L_{C}) associated with it based on the concatenated input stream. The combiner optimizer 244 is configured to train the first feature extractor sub-network 232A, the second feature extractor sub-network 232B, and the combiner optimizer together 234 by optimizing a total loss (L_{T}).

In accordance with embodiments of the present description, the training module 240 is configured to alternate between the feature optimizer 242 and the combiner optimizer 244 until a training loss reaches a defined saturation value. Thus, the training of the multi-stream neural network 230 is implemented in a round robin manner by first training the individual feature extractor sub-networks 232A and 232B individually, followed by the training the full network including the combiner sub-network 234. The individual feature extractor sub-networks may be either trained simultaneously or sequentially.

The training module 240 is configured to alternate between the feature optimizer 242 and the combiner optimizer 244 “n” times. In some embodiments, “n” is in a range from 1 to 5. In some embodiments, “n” is in a range from 2 to 4. It should be noted that when “n” is 1, the training module 240 is configured to stop the training of the dual-stream neural network 230 after the first round of training.

Once the training of the dual-stream neural network is completed, a trained dual-stream neural network is generated. The trained dual-stream neural network is configured to receive the global image stream 12A and the local image stream 12N from the receiver 210 and generate a combined feature set 24.

As shown in FIG. 5, the processor 220 further includes a differential diagnosis generator 250 configured to generate the differential diagnosis of the dermatological condition based on a combined feature set 24 generated by the trained multi-stream network.

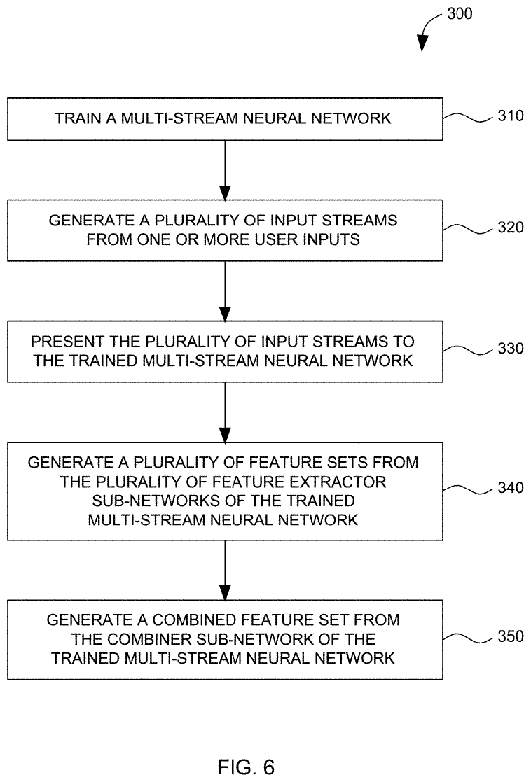

FIG. 6 is a flowchart illustrating a method 300 for generating a differential diagnosis in a healthcare environment. The method 300 may be implemented using the system of FIG. 1, according to some aspects of the present description. Each step of the method 300 is described in detail below.

At block 310, the method 300 includes training a multi-stream neural network. As mentioned earlier with reference to FIG. 1, the multi-stream network includes a plurality of feature extractor sub-networks and a combiner sub-network.

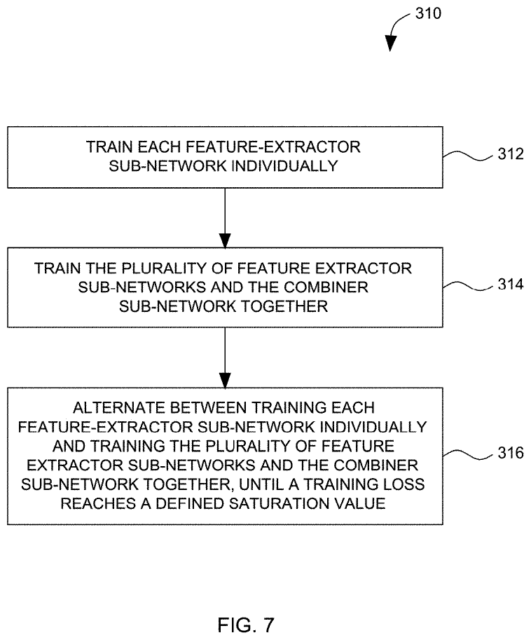

The step 310 of training the multi-stream neural network is further described in FIG. 7. At block 312, the step 310 includes training each feature-extractor sub-network individually. At block 314, the step 310 includes training the plurality of feature extractor sub-networks and the combiner sub-network together. Training of each feature-extractor sub-network individually includes optimizing each feature-extractor sub-network loss (L_{FE}), and training the plurality of feature extractor sub-networks and the combiner sub-network together includes optimizing a total loss (L_{T}).

At block 316, the step 310 includes alternating between training each feature-extractor sub-network individually and training the plurality of feature extractor sub-networks and the combiner sub-network together, until a training loss reaches a defined saturation value, thereby generating a trained multi-stream neural network. In some embodiments, step 316 includes alternating between training each feature-extractor sub-network individually and training the plurality of feature extractor sub-networks and the combiner sub-network together n times, wherein n is a range from 1 to 5. In some embodiments n is a range from 2 to 4.

Referring again to FIG. 6, at block 320, the method 300 includes generating a plurality of input streams from one or more user inputs. At block 330, the method 300 includes presenting the plurality of input streams to the trained multi-stream neural network. Further, at block 340, the method 300 includes generating a plurality of feature sets from the plurality of feature extractor sub-networks of the trained multi-stream neural network, based on the plurality of input streams. Furthermore, at block 350, the method includes generating a combined feature set from the combiner sub-network of the trained multi-stream neural network, based on the plurality of features sets. At block 360, the method 300 includes generating the differential diagnosis based on the combined feature set.

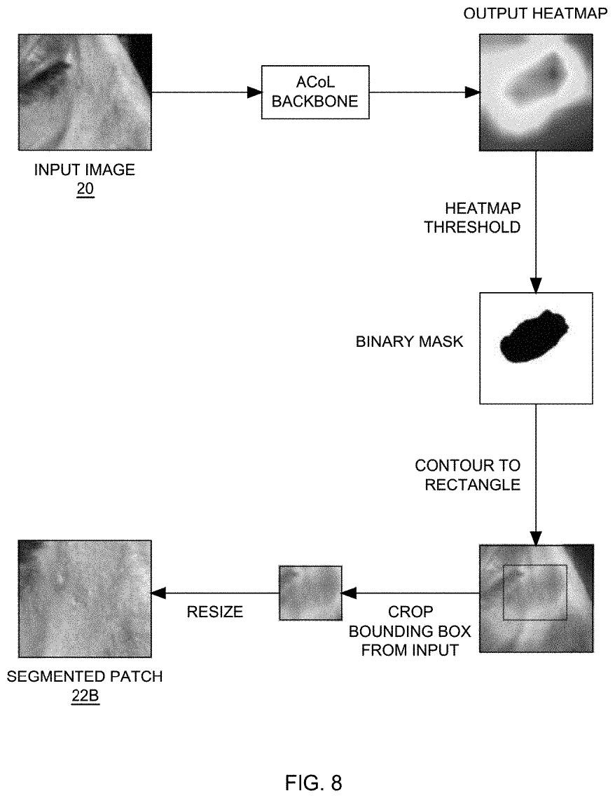

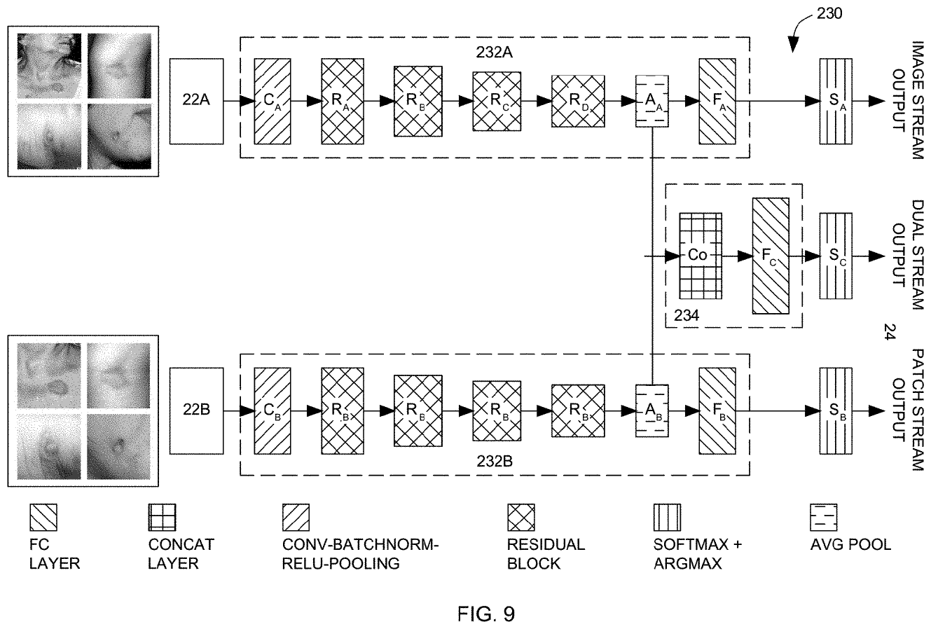

An example dual-stream neural network and a related method for generating a differential diagnosis of a dermatological condition is described with reference to FIGS. 8-9. FIG. 8 illustrates a process flow for generating a local image 22B from a user-grade image 20 of a skin-disease using weakly supervised segmentation maps. The local image 22B includes a cropped patch of region of interest (RoI) from the user-grade image 20. The RoI is the region of the image where the skin disease is highly prominent. This is considered as foreground, and the remaining region as background in the algorithm.

Assuming X to be the input user-grade image, X_{patch} refers to the patch RoI-cropped patch. ACoL, a weakly supervised segmentation technique was used to compute X_{patch}. The input image X was downsampled to a smaller size and fed to the ACol Network.

1,H=N(w,X_{downsampled}) (1)

wherein 1 is the predicted label from the ACol network for the downsampled input image X_{downsampled}, H is the heatmap of the corresponding class, N is the ACoL network and w is the model weights.

FIG. 8 shows the heatmaps obtained from ACoL. Heat maps for the images misclassified by ACoL network were also visualized. Heatmaps looked quite accurate despite the misclassification because majority of the images have a single RoI. The network had learnt that this region was important, but was unable to classify correctly as many conditions look similar to each other. To reduce the misclassification of skin conditions, it was important to analyze the features inside the RoI.

The heatmap H was normalized to a range of 0-1 and converted to a binary image X_{binary} using a threshold αt, 0<αt<1. For each pixel in H, if the pixel value was less than at, then corresponding pixel in Xbinary was assigned 0 value, otherwise it was assigned 1 value. If B is the image binarization function with at as threshold, then:

X_{binary}=B(H,αt) (2)

X_{binary} had image contours corresponding to the boundary of the skin condition of interest. A rectangular bounding box was fitted to the segmented mask, and the bounding box with the maximum area was selected as the final RoI. The RoI was cropped from full resolution input image X, resized the same size as X_{downsampled}, and was labeled X_{patch}.

FIG. 9 shows the architecture of an example dual-stream neural network D which is a composition of three sub-networks as described by equation 3 below

D(w,x)=S_{c}(w_{c},(Sᵢ(wᵢ,X_{downsampled}),Sₚ(wₚ,X_{patch}))) (3)

where Sᵢ is the image sub-network (global feature extractor sub-network 232A of FIGS. 4 and 5), Sₚ is the patch sub-network (local feature extractor sub-network 232B of FIGS. 4 and 5) and S_{c} is the combiner sub-network (combiner sub-network 234 of FIGS. 4 and 5). Sᵢ and Sₚ learn features from image and RoI-cropped patch, respectively. Sc takes the outputs from final layers of Sᵢ and Sₚ as input, and has extra layer(s) to learn from combination of these different type of features, as shown in FIG. 9. Resnet50 was used as a backbone network to both of the streams, Sᵢ and Sₚ. It has a conv-relu pool layer followed by four residual blocks, then a GAP layer and an FC layer. Input to Sᵢ was X_{downsampled} and input to Sₚ was X_{patch}. The combiner network S_{c} consisted of a concat layer and a FC layer. Concat layer vertically concatenates output from GAP layer of both the streams. A softmax layer+argmax layer was added each of the three FC layers to get the predicted labels. The output from the combiner sub-network was considered the final output of the dual-stream network.

The ground truth labels were used to calculate loss corresponding to each of the output: Lᵢ corresponding to image stream, Lₚ corresponding to patch stream and L_{c} corresponding to combiner sub-network. Since the image stream and patch stream are independent, a common stream loss Lₛ was defined as Lᵢ+Lₚ. A total loss Lₜ was also defined which combined stream loss and combiner loss as follows:

Lₜ=L_{c}+βLₛ (4)

wherein, β is a hyper-parameter called loss ratio. It balances the learning between independent feature from streams and the combined features learning. The value of β was chosen empirically.

The dual-stream neural network was optimized in an alternate manner over multiple phases. In first phase of learning, the network was optimized using stream loss Lₛ which helps it to learn independent features from the stream. When training loss stopped decreasing, the loss was switched to total loss Lₜ. In this second phase of learning, the network now learned combined features from both the streams with the help of combiner sub-network Sc, as Lₜ contains combiner sub-network's loss. When training loss stopped decreasing in the second phase, the network was switched back to optimizing Lₛ, and so on. Thus, the dual-stream neural network architecture was designed to keep on alternating between Lₜ and Lₛ until the network stopped to learn, or the training loss reached a defined saturation value. Alternating between stream loss and total loss ensured that a balance was struck between learning both independent stream features, as well as learning from correlation of these features. Better learnt independent features can lead to learning of better combined features as well. Similarly, better combined features can induce better independent features as well.

Two different data sets SD-198 and SD-75 were used to test the dual-stream neural network described herein. The SD-198 data set contained 198 categories of skin diseases and 6584 clinical images. Images in this data set covered a lot of diverse situations. These images were collected using digital cameras and mobile phones, uploaded by patients to the dermatology Dermquest website and were annotated by professional dermatologists.

The SD-75 data set contained 12,296 images across 75 dermatological conditions. Only those conditions were included in the dataset which had at least 10 images. Similar to the other user-grade datasets, this dataset was also heavily imbalanced because of varying rarity of different dermatological conditions. Images were taken by the patients themselves using mobile phones of varying camera quality. The dataset was diverse in terms of patient profiles like: age (child, adult, and old), gender (male and female). Diversity was also present in the form of: skin tone (white, black, brown, and yellow), disease site (face, head, arms, nails, hand, and feet) and lesions in different stages (early, middle, and late). The images also contained variations in orientation, illumination and scale. The images were annotated by professional dermatologists.

Both the SD-198 and SD-75 datasets were divided by randomly splitting each data set into training and testing sets with 8:2 samples. Specifically, 5268 images were selected for training and the remaining 1316 images for testing in the SD-198 dataset and 9836 images were selected for training versus 2460 images for testing in the SD-75 dataset.

A set of experiments were conducted to establish baseline network and training parameter values. Based on these experiments, Resnet50 was selected as the backbone network and binary cross entropy loss as primary loss function. The learning rate decay and decay step size were set to 0.5 and 100, respectively. A default value of 0.5 was chosen for threshold parameter at and value of β was set to 0.25.

The full network was optimized as a comparative example along with multi-phase alternate optimization. Full network optimization refers to optimizing all the sub-networks together. Multi-phase alternate optimization refers to the round robin optimization of sub-networks in accordance with embodiments of the present description. Table 1 shows the performances of dual-stream network as well as individual sub-networks using both the optimization strategies on datasets SD-75 and SD-198.

TABLE 1
The performance of image stream, patch stream and the
dual-stream network on SD-75 and SD-198 datasets

| Data Set | Stream | Optimization | Accuracy | F1 |
|---|---|---|---|---|
| SD-198 | Image | Full | 66.5 ± 1.4 | 63.9 ± 1.6 |
| SD-198 | Patch | Full | 65.2 ± 1.4 | 63.1 ± 1.7 |
| SD-198 | Dual | Full | 67.2 ± 1.3 | 66.5 ± 1.5 |
| SD-198 | Dual | Alternate | 71.4 ± 1.1 | 70.9 ± 1.2 |
| SD-75 | Image | Full | 46.6 ± 1.9 | 44.9 ± 1.9 |
| SD-75 | Patch | Full | 43.7 ± 2.1 | 42.5 ± 1.9 |
| SD-75 | Dual | Full | 47.2 ± 1.9 | 46.6 ± 1.8 |
| SD-75 | Dual | Alternate | 51.8 ± 1.7 | 50.9 ± 1.8 |

As shown in Table 1, the alternate optimization comprehensively outperformed full optimization in both the datasets.

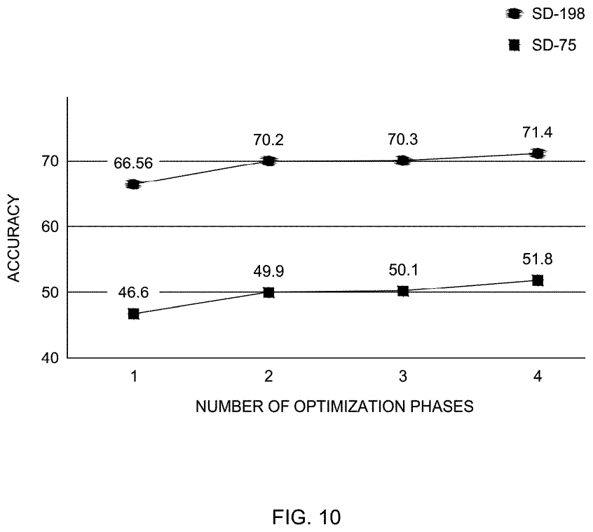

FIG. 10 is a graph showing the performance of the dual-stream neural network on both the data sets over multiple phases of alternate optimization. In FIG. 10, the network was optimized over four phases and two rounds, where phases 1 and 2 correspond to round 1, and phases 3 and 4 correspond to round 2. As shown in FIG. 10, a sharp increase in accuracy was observed in phase 2 where the training/learning was switched from individual-stream learning (training individual feature extractor sub-networks) to combined learning (training the feature extractor sub-networks and combiner network together), thus giving a boost to the network performance. Improvement in the phase 3 was not much significant, as in this phase only the individual streams were being optimized. The network learned better stream features in this phase, but overall performance did not improve. On switching back to combined learning in phase 4, a performance boost was observed again, primarily due to the better stream features learnt in the previous phase.

The systems and methods described herein may be partially or fully implemented by a special purpose computer system created by configuring a general-purpose computer to execute one or more particular functions embodied in computer programs. The functional blocks and flowchart elements described above serve as software specifications, which may be translated into the computer programs by the routine work of a skilled technician or programmer.

The computer programs include processor-executable instructions that are stored on at least one non-transitory computer-readable medium, such that when run on a computing device, cause the computing device to perform any one of the aforementioned methods. The medium also includes, alone or in combination with the program instructions, data files, data structures, and the like. Non-limiting examples of the non-transitory computer-readable medium include, but are not limited to, rewriteable non-volatile memory devices (including, for example, flash memory devices, erasable programmable read-only memory devices, or a mask read-only memory devices), volatile memory devices (including, for example, static random access memory devices or a dynamic random access memory devices), magnetic storage media (including, for example, an analog or digital magnetic tape or a hard disk drive), and optical storage media (including, for example, a CD, a DVD, or a Blu-ray Disc). Examples of the media with a built-in rewriteable non-volatile memory, include but are not limited to memory cards, and media with a built-in ROM, including but not limited to ROM cassettes, etc. Program instructions include both machine codes, such as produced by a compiler, and higher-level codes that may be executed by the computer using an interpreter. The described hardware devices may be configured to execute one or more software modules to perform the operations of the above-described example embodiments of the description, or vice versa.

Non-limiting examples of computing devices include a processor, a controller, an arithmetic logic unit (ALU), a digital signal processor, a microcomputer, a field programmable array (FPA), a programmable logic unit (PLU), a microprocessor or any device which may execute instructions and respond. A central processing unit may implement an operating system (OS) or one or more software applications running on the OS. Further, the processing unit may access, store, manipulate, process and generate data in response to the execution of software. It will be understood by those skilled in the art that although a single processing unit may be illustrated for convenience of understanding, the processing unit may include a plurality of processing elements and/or a plurality of types of processing elements. For example, the central processing unit may include a plurality of processors or one processor and one controller. Also, the processing unit may have a different processing configuration, such as a parallel processor.

The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

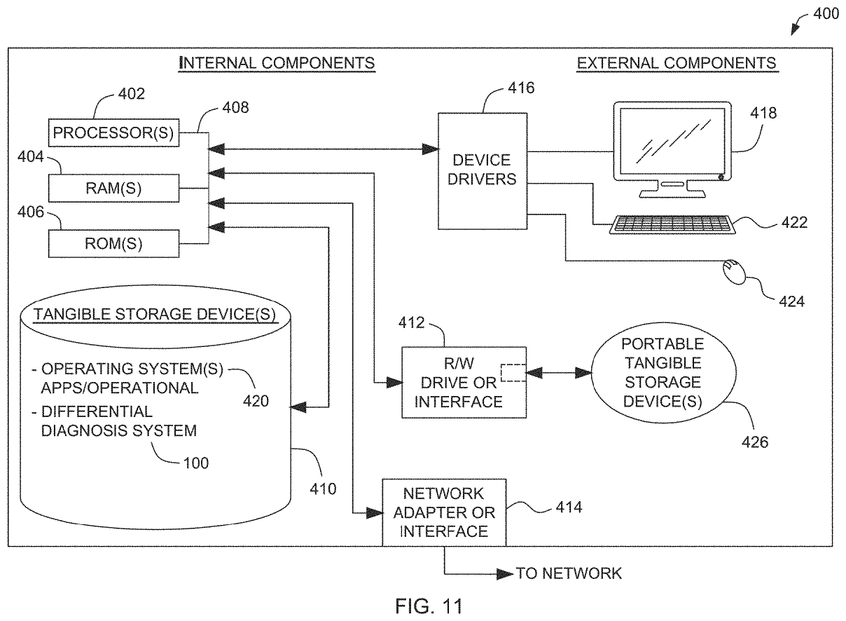

One example of a computing system 400 is described below in FIG. 11. The computing system 400 includes one or more processor 402, one or more computer-readable RAMs 404 and one or more computer-readable ROMs 406 on one or more buses 408. Further, the computer system 408 includes a tangible storage device 410 that may be used to execute operating systems 420 and differential diagnosis system 100. Both, the operating system 420 and the differential diagnosis system 100 are executed by processor 402 via one or more respective RAMs 404 (which typically includes cache memory). The execution of the operating system 420 and/or differential diagnosis system 100 by the processor 402, configures the processor 402 as a special-purpose processor configured to carry out the functionalities of the operation system 420 and/or the differential diagnosis system 100, as described above.

Examples of storage devices 410 include semiconductor storage devices such as ROM 504, EPROM, flash memory or any other computer-readable tangible storage device that may store a computer program and digital information.

Computing system 400 also includes a R/W drive or interface 412 to read from and write to one or more portable computer-readable tangible storage devices 4246 such as a CD-ROM, DVD, memory stick or semiconductor storage device. Further, network adapters or interfaces 414 such as a TCP/IP adapter cards, wireless Wi-Fi interface cards, or 3G or 4G wireless interface cards or other wired or wireless communication links are also included in the computing system 400.

In one example embodiment, the differential diagnosis system 100 may be stored in tangible storage device 410 and may be downloaded from an external computer via a network (for example, the Internet, a local area network or another wide area network) and network adapter or interface 414.

Computing system 400 further includes device drivers 416 to interface with input and output devices. The input and output devices may include a computer display monitor 418, a keyboard 422, a keypad, a touch screen, a computer mouse 424, and/or some other suitable input device.

In this description, including the definitions mentioned earlier, the term ‘module’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware. The term code, as used above, may include software, firmware, and/or microcode, and may refer to programs, routines, functions, classes, data structures, and/or objects.

Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above. Shared memory hardware encompasses a single memory device that stores some or all code from multiple modules. Group memory hardware encompasses a memory device that, in combination with other memory devices, stores some or all code from one or more modules.

In some embodiments, the module may include one or more interface circuits. In some examples, the interface circuits may include wired or wireless interfaces that are connected to a local area network (LAN), the Internet, a wide area network (WAN), or combinations thereof. The functionality of any given module of the present description may be distributed among multiple modules that are connected via interface circuits. For example, multiple modules may allow load balancing. In a further example, a server (also known as remote, or cloud) module may accomplish some functionality on behalf of a client module.

While only certain features of several embodiments have been illustrated and described herein, many modifications and changes will occur to those skilled in the art. It is, therefore, to be understood that the appended claims are intended to cover all such modifications and changes as fall within the scope of the invention and the appended claims.

1. A system for generating a differential diagnosis in a healthcare environment, the system comprising:
a receiver configured to receive one or more user inputs and generate a plurality of input streams based on the one or more user inputs, and
a processor operatively coupled to the receiver, the processor comprising:
a multi-stream neural network comprising:
a plurality of feature extractor sub-networks configured to generate a plurality of feature sets, each feature extractor sub-network configured to generate a feature set corresponding to a particular input stream,
a combiner sub-network configured to generate a combined feature set based on the plurality of features sets;
a training module configured to generate a trained multi-stream neural network, the training module comprising:
a feature optimizer configured to train each feature-extractor sub-network individually, and
a combiner optimizer configured to train the plurality of feature extractor sub-networks and the combiner sub-network together,
wherein the training module is configured to alternate between the feature optimizer and the combiner optimizer until a training loss reaches a defined saturation value; and
a differential diagnosis generator configured to generate the differential diagnosis based on a combined feature set generated by the trained multi-stream network.

2. The system of claim 1, wherein the feature optimizer is configured to train each feature-extractor sub-network individually by optimizing each feature-extractor sub-network loss (L_{FE}), and the combiner optimizer is configured to train the plurality of feature extractor sub-networks and the combiner sub-network together by optimizing a total loss (L_{T}).

3. The system of claim 1, wherein the training module is configured to alternate between the feature optimizer and the combiner optimizer n times, wherein n is in a range from 1 to 5.

4. The system of claim 1, wherein the one or more user inputs comprise a text input, an audio input, an image input, a video input, or combinations thereof.

5. The system of claim 1, wherein the one or more user inputs comprise a plurality of inputs, each input of the plurality of inputs corresponding to a different modality.

6. The system of claim 1, wherein the one or more user inputs comprise a single input and the plurality of input streams comprise different input streams corresponding to the single input.

7. The system of claim 6, wherein the single input comprises a user-grade image and the plurality of input streams comprise a global image stream and a local image stream generated from the user-grade image.

8. The system of claim 7, wherein the system is configured to generate the differential diagnosis for a dermatological condition based on the global image stream and the local image stream.

9. A system for generating differential diagnosis for a dermatological condition, comprising:
a receiver configured to receive a user-grade image of the dermatological condition, the receiver further configured to generate a global image stream and a local image stream from the user-grade image; and
a processor operatively coupled to the receiver, the processor comprising:
a dual-stream neural network, comprising:
a first feature extractor sub-network configured to generate a plurality of global feature sets based on the global image stream;
a second feature extractor sub-network configured to generate a plurality of local feature sets based on the local image stream; and
a combiner sub-network configured to generate a combined feature set based on the plurality of global feature sets and the plurality of local feature sets.
a training module configured to generate a trained dual-stream neural network, the training module comprising:
a feature optimizer configured to train the first feature extractor sub-network and the second feature extractor sub-network individually; and
a combiner optimizer configured to train the first feature extractor sub-network, the second feature extractor sub-network, and the combiner optimizer together,
wherein the training module is configured to alternate between the feature optimizer and the combiner optimizer until a training loss reaches a defined saturation value; and
a differential diagnosis generator configured to generate the differential diagnosis of the dermatological condition, based on a combined feature set generated by the trained dual-stream network.

10. The system of claim 9, wherein the feature optimizer is configured to train the first feature extractor sub-network and the second feature extractor sub-network individually by optimizing each feature-extractor sub-network loss (L_{FE}), and the combiner optimizer is configured to train the first feature extractor sub-network the second feature extractor sub-network, and the combiner optimizer together by optimizing a total loss (L_{T}).

11. The system of claim 9, wherein the training module is configured to alternate between the feature optimizer and the combiner optimizer n times, wherein n is in a range from 1 to 5.

12. The system of claim 9, wherein the receiver further incudes a global image generator configured to generate the global image stream from the user-grade image by down sampling the user-grade image, and the receiver further includes a local image generator configured to generate the local image stream from the user-grade image by extracting regions of interest from the user-grade image.

13. A method for generating a differential diagnosis in a healthcare environment, comprising:
training a multi-stream neural network to generate a trained multi-stream neural network, the multi-stream network comprising a plurality of feature extractor sub-networks and a combiner sub-network, the training comprising:
training each feature-extractor sub-network individually,
training the plurality of feature extractor sub-networks and the combiner sub-network together, and
alternating between training each feature-extractor sub-network individually and training the plurality of feature extractor sub-networks and the combiner sub-network together, until a training loss reaches a defined saturation value, thereby generating a trained multi-stream neural network;
generating a plurality of input streams from one or more user inputs;
presenting the plurality of input streams to the trained multi-stream neural network;
generating a plurality of feature sets from the plurality of feature extractor sub-networks of the trained multi-stream neural network, based on the plurality of input streams;
generating a combined feature set from the combiner sub-network of the trained multi-stream neural network, based on the plurality of features sets; and
generating the differential diagnosis based on the combined feature set.

14. The method of claim 13, wherein training each feature-extractor sub-network individually comprises optimizing each feature-extractor sub-network loss (L_{FE}), and training the plurality of feature extractor sub-networks and the combiner sub-network together comprises optimizing a total loss (L_{T}).

15. The method of claim 13, wherein the training comprises alternating between training each feature-extractor sub-network individually and training the plurality of feature extractor sub-networks and the combiner sub-network together n times, wherein n is a range from 1 to 5.

16. The method of claim 13, wherein the one or more user inputs comprise a text input, an audio input, an image input, a video input, or combinations thereof.

17. The method of claim 13, wherein the one or more user inputs comprise a plurality of inputs, each input of the plurality of inputs corresponding to a different modality.

18. The method of claim 13, wherein the one or more user inputs comprise a single input and the plurality of input streams comprise different input streams corresponding to the single input.

19. The method of claim 18, wherein the single input comprises a user-grade image and the plurality of input streams comprise a global image stream and a local image stream generated from the user-grade image.

20. The method of claim 19, wherein the method comprises generating the differential diagnosis for a dermatological condition based on the global image stream and the local image stream.
